(19) United States
(12) Patent Application Publication
(10) Pub. No.: US 2026/0269052 A1
(43) Pub. Date: Sep. 10, 2026

(54) METHOD AND DEVICE FOR ANATOMOPATHOLOGY WITH OBJECT IDENTIFICATION AND TRACKING

(71) Applicant: ALGOSCOPE, Compiègne (FR)

(72) Inventor: Soufiane Zakaria AZDAD, Compiègne (FR)

(73) Assignee: ALGOSCOPE, Compiègne (FR)

(21) Appl. No.: 19/167,188

(22) PCT Filed: Mar. 21, 2024
(86) PCT No.: PCT/EP2024/057589

§ 371 Date: Sep. 19, 2025

(30) Foreign Application Priority Data
Mar. 22, 2023 (FR) FR2302709

(51) Int. Cl. G16H 30/20; G01N 1/06; G01N 1/30; G16H 10/40; G16H 10/60; G16H 40/40

(52) U.S. Cl. CPC G16H 30/20; G01N 1/06; G01N 1/30; G16H 10/40; G16H 10/60; G16H 40/40

(57) ABSTRACT

A method for histopathologically examining biological tissues, in particular surgical specimen or biopsy, in particular for anatomo-cytopathology ACP, the method comprising identifying and tracking object by analysing a video stream during a pre-analytical step and/or an analytical step for the biological tissues.

### TECHNICAL FIELD

The invention concerns pathological anatomy and cytology.

### STATE OF THE ART

Pathological anatomy and cytology, or anatomo-cytopathology (ACP) refers to the medical speciality that studies tissues, cells, and their abnormalities, to help diagnose diseases, e.g. tumour diseases, or diseases with inflammatory, degenerative, vascular, metabolic or infectious cause. Anatomo-cytopathology also refers to autopsy and fetopathology procedures.

Anatomo-cytopathology (ACP) is a medical speciality that is at the crossroads of clinical training, imaging and biology. It may only be performed in France by doctors qualified in the ACP, or by medical laboratory technicians, under the responsibility of a medical biologist or a doctor qualified in the ACP (article L4352-1 of the French Public Health Code).

Pathological anatomy and cytology implements macroscopic study, as well as various microscopy, molecular biology methods for histological and cytological analysis.

ACP diagnosis is based on the presence or absence of macroscopic or microscopic tissue or cellular abnormalities relative to normal. ACP diagnosis is essential for tumour diagnosis and personalised therapeutic management and targeted treatment of cancers.

The anatomopathologist analyses collected samples for histopathological examination: these are biopsies, curettage products, endoscopic resections and aspirations, spontaneous expulsion equipment, surgical or amputation specimens, organ samples. The examination may take place in a pathology laboratory, or during surgical procedure (extemporaneous examination).

“Biopsy” here means a small piece of tissue collected transcutaneously or endoscopically, for example a tumour sample.

“Surgical specimen” here means partial or complete exereses of one or more organs, collected in the operating theatre during a surgical procedure.

Examination of the surgical specimens comprises macroscopic examination allowing their measurement, weighing and description. Surgical specimens have to be fixed, dissected, described, measured, and sampled according to specific protocols.

Fixation should occur within a short period of time: otherwise, tissue desiccation or autolysis may make the study difficult or even impossible. Prior to fixation, the hollow organs should be opened and if necessary cleared of their contents, in order to prevent mucosal autolysis. Solid organs such as liver and spleen should be cut into slices to facilitate smooth and rapid penetration of the fixing agent.

Macroscopy makes it possible in particular to notice the appearance, colouration, size and consistency of an organ or a mucous membrane, suggestive of a pathology, for example hepatic steatosis, cholestasis, ulcerative colitis.

Digital images can be acquired during macroscopy or subsequent microscopy steps, to be indexed and saved in a laboratory management system (LMS). Images of the surgical specimen, fresh or fixed, which will be taken apart by dissection, are stored digitally and attached to the patient record.

Macroscopy laboratory workstations conventionally include a stainless steel table on which a dissecting board is placed. In some cases, the workstation is provided with an imaging device for taking images and annotating these images during macroscopy.

In the macroscopy phase, the technician usually has one or more vials labelled with a record number specific to the sample, and linked to the patient in the laboratory management system. The technician extracts the contents of the vial and places it in a cassette or cassettes that are also labelled.

Example cassettes are set forth in documents U.S. Pat. No. 11,774,330 (Biopath, 2023), EP2316010B1 (The Univ of Miami, 2022), U.S. Pat. No. 8,877,146 (Biopath, 2014), U.S. Pat. No. 7,771,992 (Leica Biosystems, 2010), DE4306233 (Guenter, 1994), GB2278441 (Cellpath, 1994).

For the medical laboratory technician, manual operations are repetitive and carried out in large numbers, every day. This leads to risk of errors, bottle mix-ups and omissions can occur.

The risk of error, mix-up or omission is also possible during the other phases of preanalysis, which begins with collecting from the patient and ends with reading of the slide by the pathologist, such as during cryotomy, decalcification, dehydration, embedding or inclusion, microtomy, spreading and staining.

Sagar et al. (Laboratory errors in pathology and troubleshooting methods, Journal of Pharmaceutical Negative Results, 2022 Special Issue 7, Vol. 13, p272-278. 7p. DOI: 10.47750/pnr.2022.13.S07,037) set forth a list of human errors that can occur during pre-analytical, analytical and post-analytical steps.

Dimenstein (Root cause analysis of specimen misidentification in surgical pathology accession and grossing, 2006, DOI: 10.1309/B7KJB4QDM7YE35CV) sets forth various sources of human errors, e.g. confusion between right and left, typing error, incorrect choice of vial or cassette, these errors being seen as related to human psychology, and therefore unavoidable.

Santana et al. (Errors in surgical pathology laboratory, 2018, DOI: 10.5772/intechopen.72919), based on a summary of forty articles, conclude that there is an inevitable risk of human errors during anatomo-pathological laboratory analyses, wherein these errors can have serious consequences, generating false positives or false negatives.

Reference may also be made to documents Simo Huang et al. (Errors encountered in the diagnostic path: A prospective single-institution study, Journal of Cutaneous Pathology, 2023, DOI/10.1111/cup.14474), Steelman, Surgical Specimen Management: A Descriptive Study of 648 Adverse Events and Near Misses, Arch Pathol Lab Med, 2016, DOI 10.5858/arpa.2016-0021-OA), Tang et al. (Patient safety issues in pathology: from mislabeled specimens to interpretation errors, 2018, doi. org/10.5772/intechopen.796334), Van Moll et al. (The Nature, Causes, and Clinical Impact of Errors in the Clinical Laboratory Testing Process Leading to Diagnostic Error: A Voluntary Incident Report Analysis, Journal of Patient Safety, 2023, 19(8), 573-579).

Anatomopathology laboratories are prone to sample identification errors due to the numerous steps of sample modification.

Several different stakeholders must ensure that the identification of the sample is preserved during different steps of transforming the sample, including collection, macroscopic description, cassette placement, inclusion in a block (usually paraffin), slide transfer after cutting the paraffin block with microtome.

An identification error can occur at any of these steps, and can have serious consequences for the patient, such as delayed diagnosis of a disease (in case of a false negative), or unnecessary surgery (in case of a false positive).

Various empirical means are implemented to try to prevent the risks of identity errors on the samples. For example, it is recommended to avoid recording and subsequently describing similar samples. It is also recommended to minimise absent-mindedness during transcription or sample identity verification.

In an attempt to reduce the risk of errors, containers such as vials or jars containing tissue samples delivered to the anatomopathology laboratory carry two different identifiers, namely the patient's first and last name and a patient-specific identification number on the other hand. The date of collection may also be indicated on each container.

Standard ISO 15189-12 requires that all samples received are recorded in an admission register, in a computing system, with the date and time of receipt of the samples to be recorded. During this registration, an identification number is assigned to each sample received, and this identification number is retained throughout the sample processing in the anatomopathology laboratory. The 2022 version of standard ISO 15189 states that each ACP review request must be considered as a contract, and must ensure traceability of the patient, request and prescriber. By traceability, standard ISO 9000:2005 refers to the ability to find the history, implementation or location of what is being examined.

Anatomo-cytopathology is a speciality practised by a restricted number of professionals. In France, according to the federation of pathologists, 1,471 doctors practised in 2018. According to the AFAQAP (Association Française d'Assurance Qualité en Anatomie et Cytologie Pathologiques [French Association for Quality Assurance in Pathological Anatomy and Cytology]), there were around 270 ACP structures and laboratories in France in 2020, including 130 freelance structures and 135 hospital structures.

The activities of anatomo-pathology professionals are separate, in technical and regulatory terms, as well as for the machines used, the activities of medical analysis laboratory technicians. In particular, the workflow of a ACP laboratory includes, for tissue collected samples, specific steps of collected sample fixation, macroscopy, cassetting, embedding (usually in paraffin), cryostat or microtome cutting. Each of these steps can result in errors and loss of traceability.

In an attempt to reduce the risk of identification errors, and to improve traceability, thermal printing systems, on cassettes or slides, alphanumeric characters and two-dimensional barcodes are marketed. For example, refer to documents IT201900012333 (Logibiotech, 2021), US2019324048 (Sakura Finetek, 2019), WO2014018114, (Aperio technologies, 2014), WO2007/078842 (Ventana Medical Systems, 2007). Reference may also be made to Mei-Hua et al. (The Effect of Barcode Technology Use on Pathology Specimen Labeling Errors, 2019, doi 10.1002/aorn.12585), Pantanowitz et al. (Tracking in Anatomic Pathology, 2013, Arch Pathol Lab Med, DOI 10.5858.arpa.2013-0125-SA). Such systems reduce the risks associated with manual registration, but do not eliminate input errors in a computing system, and handling errors.

Norgan et al. (Radio-frequency identification specimen tracking to improve quality in anatomic pathology, Arch Pathol Lab Med—Vol 144, February 2020, DOI: 10.5858/arpa.2019-0011-OA) describe the use of high-frequency (902-928 MHz) passive RFID tags to reduce risk of errors during pre-analytical steps, the RFID tags being placed on a carrier bearing a unidirectional barcode (coding the patient's identification number), and a bidirectional barcode (coding the identifier of the RFID tag). At the end of a 6-month observation period on a total of 56,000 samples, the authors concluded that there was a reduction in identification errors compared to using bar codes alone. There are many drawbacks to using RFID tags. The cost of RFID tags is high. Interference risks during reading cannot be eliminated.

Document WO2022/266459 (Bioptek, 2022) describes a system and a method for automated processing of biopsy samples, and discusses still image analysis.

### GENERAL DESCRIPTION OF THE INVENTION

One of the purposes of the invention is to reduce risks of error during anatomical and cytological pathology work.

One of the objects of the invention is to provide a method and a device ensuring traceability of a collected sample in anatomopathology, from the operating theatre to cutting and spreading on slides and archiving of slides and paraffin blocks.

The invention aims to ensure traceability over all the steps of the ACP, whether it is the pre-analytical phase or the analytical phase, and advantageously the post-analytical phase.

Analytical phase refers to all steps in the chronological order of work on biological material (human or animal), the collection or the sample, from the analysis itself to its technical and/or medical validation.

The pre-analytical phase refers to all the steps in chronological order starting with formulating the analysis request by the prescriber, identifying the patient, collecting the biological material, conveying it to the ACP laboratory, receiving it at the ACP laboratory, recording or encoding it and any other operation or handling of this material before the start of the analytical phase.

Post-analytical phase refers to all the steps in chronological order from the end of the analytical phase, including review of the results, release authorisation, preparation of the report, transmission of the validated results to their recipients, storage, archiving and/or disposal of biological material.

The storage (including archiving and organisation thereof) of biological material, including partial samples and residual material, is the final responsibility of the operator of the ACP laboratory.

Paraffin blocks should be preserved: they may be useful subsequently or necessary, in order to refine or supplement the diagnosis of the patient from whom they originate, for example in the event of an additional immunohistochemical or molecular examination.

Histological sections and stained cytology slides, whether analogue or digital, may be subject to subsequent interpretation and should also be preserved.

The legal minimum storage periods for biological materials vary from one State to another, and may be, for example, ten years for paraffin blocks and twenty years from the date of collection for histological sections and stained cytology slides.

The invention also aims to ensure traceability on the preservation of biological materials.

To these ends, there is provided, according to a first aspect, a method for histopathologically examining biological tissues, especially surgical specimen or biopsy, in particular for anatomo-cytopathology ACP, the method comprising identifying and tracking object by analysing a video stream during a pre-analytical step and/or an analytical step for the biological tissues.

By identifying and tracking object by analysing a video stream, it is especially meant herein computer vision techniques implementing video image segmentation or object segmentation or detection, for example active contour methods using statistical criteria. Object detection here means the detection and classification in images of objects belonging to sets of predefined categories. For example, object detection is performed in still images, by deep learning such as R-CNN. Object detection is advantageously carried out by analysing a video stream, object detection and tracking being for example carried out using YOLO.

Advantageously, the video stream is derived from at least one digital camera recording at the wavelengths perceptible by the human eye.

Alternatively, the camera records wavelengths in the infrared range. The camera can thus provide information about the surface temperature of identified objects.

Alternatively, the camera records wavelengths in the ultraviolet range. Thus, the camera can provide information about the presence of markers for biological tissue, for example on the margins of a surgical specimen.

In some implementations, at least one camera is a so-called proximity camera, and is placed as close as possible to the zone in which the work carried out on a pre-analytical workstation and/or an analytical workstation and/or a post-analytical workstation occurs.

Alternatively, at least one camera is placed so as to film the entirety of an analytical or pre-analytical workstation, this camera being for example fastened to the ceiling of a room. Advantageously, the video streams of this camera are analysed in parallel and simultaneously with the analysis of the video streams of the proximity camera (or the proximity cameras).

Object tracking is advantageously carried out in real time, i.e. at a speed adapted to the progress of each pre-analytical step and/or each analytical step.

An error message is thus advantageously generated, in the event of a discrepancy or deviation between the state of an object and a reference state for this object. The state of an object here means, for example, the open or closed situation of a container, an amount of liquid in a container, information present on a support.

In various implementations, the pre-analytical step is selected from the group comprising collecting biological tissues from the operating field, orienting biological tissues, labelling the biological tissues collected, placing the tissues in a container in a fresh state, immersing the biological tissues collected in a preservative liquid such as a fixing agent, preparing a histopathological examination request form, transporting the collected sample to a recording station, recording the collected sample in a laboratory management system.

According to various embodiments, object identification and tracking is performed by analysing a video stream during at least one of these pre-analytical steps, the object identified and tracked being for example a biological tissue collected, a container (bucket, vial) in which a biological tissue has to be placed, an examination request form, a barcode label to be fastened to a container.

Advantageously, the method comprises a step of labelling the biological tissues with generating a first patient identity, in alphanumeric form or in coded form, in particular by barcode, identifying and tracking an object by analysing a video stream comprising a step of reading this first patient identity, in particular barcode reading, or character recognition (OCR).

The barcode can be one-dimensional or two-dimensional, for example QR Code or Data Matrix.

Advantageously, the method comprises a step of preparing n advantageously digital histopathological examination request form generating a second patient identity, identifying and tracking an object by analysing a video stream comprising a step of reading this examination request form and the second patient identity, an alert message being generated in the event of a discrepancy between the first and second patient identity.

Advantageously, the method comprises a step of querying the computerised patient record and a step of collecting information elements contained in the computerised patient record, for copying into a histopathological examination request form generated in digital form.

Advantageously, the method comprises identifying and tracking object by analysing a video stream when immersing biological tissues such as a surgical specimen in a preservative liquid, such as a fixing agent, an alert message being generated when the volume of the preservative liquid is not adapted to the volume of the biological tissues.

Advantageously, the method comprises a check step performed in a buffer zone in which a plurality of collected samples of biological tissue, in particular derived from different operating rooms of an operating theatre, are gathered, the method comprising identifying and tracking object by analysing a video stream for counting containers placed in the buffer zone.

Advantageously, the method comprises identifying and tracking object by analysing a video stream during recording a collected sample in a laboratory management system, for counting the containers and/or checking suitability of the fixing agent volume for the collected sample placed in a container.

Advantageously, the method comprises identifying and tracking object by analysing a video stream during a step of preparing biological tissues selected from the group comprising dehydrating, embedding in a material, in particular paraffin, cutting especially with a cryostat or microtome to form ribbons, spreading the ribbons on supports such as glass slides, dispensing.

Advantageously, the method comprises reading the entirety of the cassette identifiers, these identifiers being alphanumeric or coded, especially in a barcode. In some implementations, for example, 50 barcodes are read for each still image.

Advantageously, at least one interface screen of a dehydration machine is filmed, an analysis of a video stream making it possible to generate information comprising, for example, the duration of the dehydration cycle, the time remaining until the end of the dehydration, the type of cycle, the presence of an alarm.

Advantageously, the method comprises a step of generating an alert message, when an operation alarm has been detected for the dehydration machine, when analysing the video stream from the camera filming the interface screen of the dehydration machine.

Advantageously, the method comprises identifying and tracking object by analysing a video stream during a step of embedding biological tissues in an embedding material such as paraffin, analysing the video stream being able to ensure counting of the biological tissues poured into the embedding mould.

Advantageously, the analytical step is chosen from the group comprising the different macroscopic study steps, especially inking the lesion margins, making samples, by sections in a human tissue collected sample, or by recutting in an animal tissue collected sample.

Advantageously, the method comprises analysing the video streams allowing counting of the samples present on the stained slides, from a cassette, and a step of verifying correlation of this number with the number of collected samples present in the cassette, or a step of verifying size of the collected samples (for example biopsy cores) and/or the surface of the collected samples, in particular to verify that no loss of material has occurred.

There is provided, according to a second aspect, an anatomopathology method comprising a step of introducing a tissue into a first container provided with a closure means, a step of closing the first container after introducing the tissue, a step of placing on the first container an identifier of the tissue introduced into the first container, a step of opening the first container and extracting the tissue from the first container, a step of moving at least one part of the tissue extracted from the first container into a second container, the second container bearing an identifier of the part of tissue, the method comprising identifying and tracking objects by analysing a video stream, the opening of the first container being detected during this identification and tracking of objects, the method comprising a step of detecting and reading the identifier placed on the first container and the identifier placed on the second container, an error message being generated in the event of a discrepancy between the identifier of the first container and the identifier of the second container.

The method makes it possible to alert the technician of a concordance loss during a step of moving the biological tissue from a first container such as a vial or a jar, to a second container such as a cassette.

Advantageously, the method comprises a step of recording the instant at which opening of the first container is detected. This recording improves traceability of the method, as the instant of detecting opening of the first container can be compared with the instant at which collection has been performed, for example by exeresis or biopsy. If fixation is to be performed for biological tissue, it can thus be verified that fixation occurs within a short period of time, preventing tissue desiccation or autolysis.

Advantageously, the second container being a cassette, closing the cassette and closing the first container are detected by identifying and tracking objects when analysing the video stream. This allows an error message to be generated if a new container is open before the first container is closed.

Advantageously, displaying the objects identified when analysing the video stream is carried out, the shape of the objects identified being represented for example by a bounding window. The operator can thus verify that the identification of the objects corresponds to the operations carried out. The operator can add, if appropriate, comments by voice command, using a keyboard, mouse or even touch screen.

There is provided, according to a third aspect, a device for implementing a method as set forth above, the device comprising means for identifying and tracking objects by analysing a video stream from at least one camera.

There is provided, according to another aspect, an anatomopathology device for implementing a method as set forth above, the device including a workstation comprising a dissecting board (cutting board) for supporting a first container provided with a closing means and a second container, the device comprising means for identifying and tracking objects by analysing a video stream, these means being able to detect opening of the first container, the device comprising means for detecting and reading an identifier placed on the first container and an identifier placed on the second container, the device comprising means for generating an error message in the event of a discrepancy between the identifier of the first container and the identifier of the second container.

Advantageously, in some implementations, the cutting board comprises a recess capable of housing a first or second container, the first container being for example a vial, the second container being for example a cassette.

Advantageously, the detection and reading means comprise a one-dimensional or two-dimensional barcode reader, for example of the QR code or Data Matrix, or even Aztec code type. Alternatively or in combination, character recognition (OCR) is performed. Advantageously, decoding the barcode is directly carried out via an image captured by a camera.

Data Matrix here refers to data coding in a cell matrix, the code being in the form of a square or rectangular symbol. The Data Matrix code is described in standard ISO/IEC16022. An encoding algorithm combines the useful data with error correcting codes. The coding format is advantageously ECC200 (Error Checking and Correcting).

The QR code is defined by standard ISO/IEC 18004. It also has a multi-level error correction system, for reconstructing missing data, when part of the code cannot be read. In some implementations, the QR code is static and advantageously integrates an URL (Uniform Resource Locator). In other implementations, the QR code is dynamic, and incorporates a short URL, redirecting the user to another URL.

The aztec code is described in standard ISO/IEC 24778.

The identifier can be placed on the containers by label adhering, thermal transfer, or by engraving, in particular laser engraving.

Advantageously, the workstation comprises weighing means. It is thus possible to compare the weight of the biological tissue, measured during biopsy or surgical exeresis, with the weight of the tissue arriving in the anatomopathology laboratory. It is also possible to verify that the amount of formol (or other preservative used) is adapted to the collected sample to be analysed.

Advantageously, the workstation comprises measuring means along several axes.

Advantageously, the device comprises a biological tissue embedding station to form a block, for example of paraffin, a station for cutting the block into thin slices with a microtome, the slices having for example a thickness in the order of 3 to 5 microns, and a station for spreading the slices on slides, the device comprising means for printing an identifier of the biological tissue on the block and on the slides.

In some implementations, the embedding station comprises one or more cameras and object identification and tracking is performed by analysing a video stream, for the operations performed at the embedding station.

In some implementations, the station for cutting into slices with a microtome comprises one or more cameras and object identification and tracking is performed by analysing a video stream, for operations performed at the cutting station.

### DESCRIPTION OF EMBODIMENTS

Other objects and advantages of the invention will appear in light of the description of embodiments, made hereinafter with reference to the appended figures wherein:

FIG. 1 is a view of a macroscopy laboratory workstation, a closed vial, an open cassette, a closed cassette, and a clamp being placed on the workstation tray;

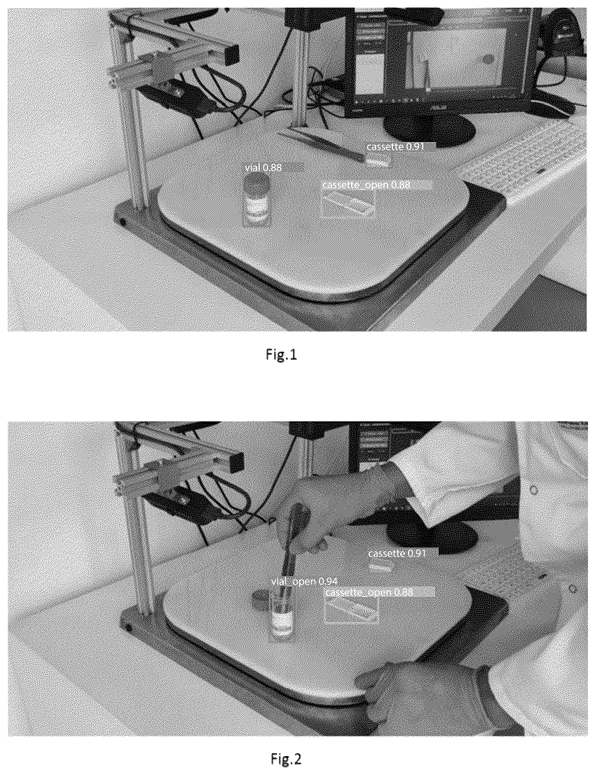

FIG. 2 is a view analogous to FIG. 1, after opening the vial, a technician collecting from the vial, using the clamp, a fragment of tissue, for example a biopsy;

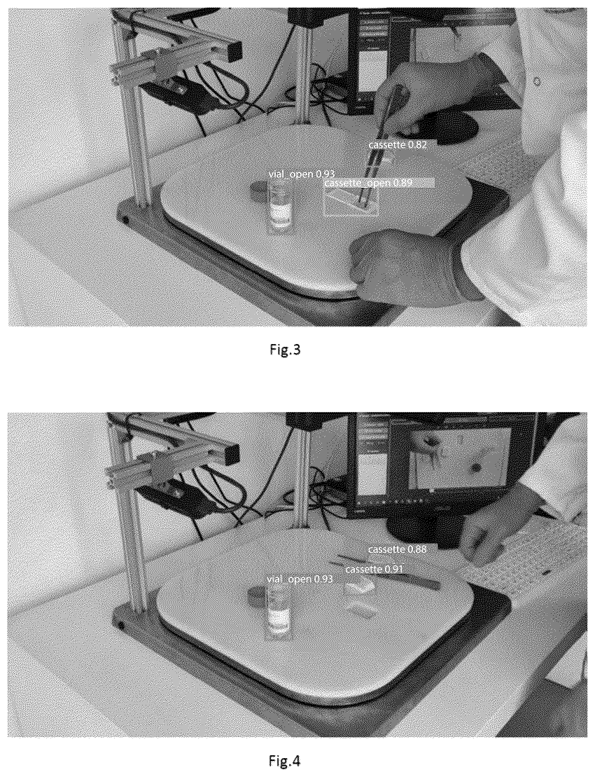

FIG. 3 is a view analogous to FIG. 2, the technician placing the tissue fragment from the vial into the open cassette;

FIG. 4 is a view analogous to FIG. 3, after closing the cassette containing the tissue fragment.

FIGS. 1 to 4 represent a macroscopy laboratory workstation.

In some embodiments, the workstation comprises a board of polymeric material, such as polyamide, polyoxymethylene (POM), or high density polyethylene (HDPE).

For example, the dissecting board (cutting board) is of mass-coloured polyethylene, the colour used corresponding to a predetermined use.

In some implementations, the board made of polymeric material includes a gauge, for example of stainless steel.

Advantageously, the board includes at least one recess, the dimensions and shape of which are such that an object such as a cassette or a vial, placed in the recess, is held in position.

Advantageously, the board is of hard, washable material for prolonged use. In some applications, the board is single-use and disposable.

Advantageously, the board is of polymeric material and is laid on a stainless steel plate, which serves as a support thereto.

Cameras film the cutting board and elements laid on the board. In some implementations, the cameras are of the RGB (Red-Green-Blue) digital camera type.

The cameras are connected to a central processing unit or server with means for analysing and processing video streams from the cameras.

Advantageously, a screen makes it possible to view all the steps of the treatment.

An image processing algorithm detects presence of objects on the cutting board and identifies these objects, as they appear on the figures.

The shape of the identified object is advantageously represented by a bounding window, as it appears in the figures.

In other implementations, the shape of the object is represented by a point, located in the centre of the object, or in the form of a contour, an outline.

Detecting objects, such as vials, cassettes, can be ensured by detecting points of interest, or by background subtraction, or image segmentation, or by supervised classification.

The detection of points of interest is for example ensured by a Moravec algorithm, or a Harris detector, a Kanade-Lucas-Tomasi detector, a SIFT (Scale Invariant Feature Transform) detector.

A presentation of the detector developed in 1977 by Hans P Moravec, and the detector developed in 1988 by Harris and Stephen, as well as a description of the SIFT method is given by Garcia (Suivi d'objets d'intérêt dans une séquence d'images: des points saillants aux mesures statistiques, thèse 2008).

The detection by background subtraction is achieved for example by modelling the colour intensity of each pixel by a Gaussian distribution.

The detection by image segmentation is for example achieved by a Mean shift algorithm.

The detection by supervised learning is for example achieved by adaptive boosting (AdaBoost), or by a support vector machine.

Tracking the identified objects is achieved, for example, by point tracking, by a probabilistic method or by core tracking. Object tracking methods by video stream analysis are set forth by Maggio et al. (Video tracking, theory and practice, 978-0470749647, 2011).

Processing the video stream is thus advantageously carried out by an artificial vision (or computer vision) system, i.e. an artificial intelligence system for analysing and processing the video streams obtained by an image acquisition means such as a camera, and to ensure object tracking.

An example of processing the video stream will now be described, some steps of this processing corresponding to appended FIGS. 1 to 4.

In a first step, the technician creates a new record for collecting samples.

This creation of a new record is advantageously linked to an identifier, for example a barcode, in particular a two-dimensional barcode, especially of the Data Matrix or QR code type, or even an Aztec code.

The identifier is advantageously a unique identifier associated with a single individual. Preferably, a unique identifier is associated with a collected sample. A patient who has undergone multiple surgeries is assigned a different identifier for each of the surgical procedures.

In some implementations, the workstation comprises a barcode reader connected to the server. Such readers are sometimes referred to as scanners or pistol grip. In other implementations, the workstation comprises a camera connected to the server, an application, for example stored on the server, for reading a barcode scanned by the camera.

Communication between the barcode reader and the server can be wired or wireless, in particular by protocols known under the trademarks Wifi, Bluetooth, Sigfox, Zigbee, Lorawan, Z-Wave, or via Near Field Communication (NFC).

At the end of this first step, the workstation is for example as represented in FIG. 1. A vial with the barcode used to open the record is laid closed on the cutting board. A clamp, an open cassette and a closed cassette are also laid on the board.

In a second step, the technician opens the vial laid on the cutting board, the vial containing the collected sample to be analysed.

The application detects that the bottle is open, via the image processing algorithm.

The technician is notified by the application that the vial is open, as it appears in FIG. 2.

The date of opening the vial is advantageously recorded in a database.

In a third step, the technician deposits the cap on the cutting board. The algorithm identifies on the video stream the location of the cap and reads the associated barcode (e.g. of the Data Matrix type).

If the cap is missing or if the associated barcode is not in concordance with that scanned, an error is reported to the user, who can then change the vial and place the correct one. The user can also ignore the error, in case of an application bug.

Advantageously, a photo is automatically taken by the device to trace opening of the vial and the record number. Advantageously, this photograph is also recorded in a database.

In a fourth step, the technician deposits a cassette in the intended location on the cutting board.

In some implementations, the cassette is of polymeric material, such as acetate polymer. The cassette comprises perforations, facilitating liquid circulation and drainage during impregnation steps. The cassette consists of a cover, being attached or hinged. In other implementations, especially when the collected sample comes from a biopsy, the cassette is closed by a wire mesh. Advantageously, the cassette is coloured, the colour being associated with a type of collected sample.

Advantageously, the application automatically detects presence of the cassette and its number readable via a bar code (for example of the Data Matrix type).

If the cassette number is different from the vial number, an error is reported to the user. In this case, they can change the cassette and put the one with the correct number.

Advantageously, a photo is automatically taken by the device, to trace the number of the cassette. Advantageously, the photograph is also recorded in a database. The workstation is in a state as for example represented in FIG. 3.

In a fifth step, when the technician finishes pouring the contents of the vial into the cassette, a photo of the contents of the cassette is advantageously taken.

The photography command can be performed by the technician by clicking with a computer mouse, or by voice command or foot command.

Advantageously, the photograph is recorded in a database.

Advantageously, automatically counting the biopsies contained in the cassette is performed.

Alternatively or in combination, automatically measuring the biopsy fragments or cores contained in the cassette is performed.

In a sixth step, the technician closes the cassette. The workstation is then in a state as represented in FIG. 4.

Advantageously, if the technician does not close the cassette, they cannot create a new record.

In a possible seventh step, a new cassette is deposited on the cutting board, a vial corresponding to several cassettes. Advantageously, this new cassette is automatically detected with its number, via its barcode (for example of the Data Matrix type). Advantageously, the same steps as those described above are implemented.

In an eighth step, the technician should close the vial. Advantageously, the application automatically detects closing the vial.

Advantageously, the time of closure of the vial is recorded in a database.

Advantageously, if another vial is opened at the same time as the first, an error is reported to the user who is thus assured that only one vial is open on the board, for any given instant.

In a possible nineth step, if a new vial associated with the same record is opened by the technician, the same steps as those described above are implemented.

Advantageously, if the technician tries to process a cassette automatically, while opening of the vial has not been detected, an error is reported to the user, notifying them to actually open the vial in the zone covered by the cameras.

Advantageously, if the vial has not been closed in the field of view of the cameras, any new vial opening will not be effective until the vial closure has been performed in the appropriate zone.

In some implementations, the user can ignore errors reported by the application, especially in the event of an application bug. Any error ignored is advantageously logged in the application.

All the recorded information is advantageously available on a screen of the workstation.

Changes/deletions can be made manually by the user, especially in the event of incorrect information provided by the application. This may concern, for example, the presence of an extra line of vial openings, an incorrect record number or incorrect counting performed by the automatic processing of cassette analysis.

Processing of video streams can be performed by an application present on a computer or a server of the workstation.

Alternatively, video stream processing and image processing algorithms are executed on a remote server.

Error management can be performed by the technician present at the macroscopy station.

Alternatively, error management can be performed remotely by another technician or supervisor.

Advantageously, the macroscopy station comprises means for weighing and measuring along several axes (in particular the three major axes of a surgical specimen).

Advantageously, the macroscopy station comprises means for surface reconstructing a surgical specimen laid on the board.

Advantageously, a central processing unit controls all the electronic boards of the macroscopy station.

The central processing unit can be that of a computer or a server. Data produced by the macroscopy station can thus be communicated to a mobile or fixed communication terminal, especially belonging to a laboratory management system (LMS). Data produced by the macroscopy station can also be accessed via an internet platform.

Data produced by the station, especially photographs, videos, surface reconstructions, can be shared in real time or not, for remote assistance, telepathology, or for training needs. For training purposes, tutorial videos, showing the macroscopic examination step by step, may advantageously be broadcast.

In some implementations, measurements, photographs and scans can be viewed on a touch screen connected to a central processing unit.

Advantageously, a software application makes it possible to annotate the photographic views taken, or the scans performed. Annotated images, scans and videos, as well as reports, can be stored in a database.

The station allows telepathology and extemporaneous telediagnostic examinations, with a technician present on site and sending digital photographs and videos to the pathologist. The pathologist may advantageously take remote control of the station, in particular to view the macroscopic image, indicate to the technician the zone to be collected.

Data produced by the station can be used as a basis for laboratory reports.

Archiving data produced by the station makes medical data and equipment that can be used for medical, scientific or forensic purposes available.

Advantageously, the precise morphometry of the surgical specimen is preserved, as well as the trace of the pre-analytical steps.

Data provided by the macroscopy station are advantageously coupled to the digital slides or virtual slides.

In some implementations, a software application makes it possible to place macroscopic images or images derived from digital slides in the volume delimited by the surface reconstruction.

In some implementations, the digital slides are produced by a slide scanner. Slide scanners are marketed, for example, under the denomination Panoramic 1000 by the company 3D Histech. Physical slide handling and loss of time searching for these slides can thus be avoided.

The method and the macroscopy station according to the invention advantageously enable tracking of all the steps through which the surgical samples pass, from surgical exeresis to microscopy slides, and their archiving, tracking thus allows identification of operations performed at the slide (or paraffin block) archiving station, making it possible to identify and date deposits and withdrawals made.

A quick presentation of these different steps is given for example by Altaleb (Surgical Pathology, ISBN 978-3-030-53689-3, 2021, see in particular page 41).

The method according to the invention makes it possible to ensure traceability of the collected samples (for example a surgical specimen or a biopsy), from the operating table until the end of the treatment of the anatomo-pathology laboratory.

In some situations, the operating table is placed in an operating room of an operating theatre, where collecting samples is normally performed by a surgeon.

In other situations, the operating table is placed in an interventional radiology room, with the sample collection (for example, a breast biopsy) being performed by a radiologist or surgeon.

In other situations, the operating table is placed in an interventional room equipped with imaging resources (interventional hybrid room in the SIBO operating theatre), the sample collection being in principle carried out by a surgeon, for example a vascular surgeon.

The medical team working in the operating room or in a SIBO hybrid room usually comprises a surgeon, an anaesthetist, an operating theatre nurse (IBODE). The surgeon may be accompanied with assistant surgeons. Most often, the anaesthetist is assigned to the operating theatre and takes care of several patients in different rooms of the theatre. The same applies to the operating theatre nurse, who can be a surgical technician, an operating assistant, a surgical or circulating assistant, or an anaesthesia-resuscitation nurse. In France, the operating theatre nurse ensures traceability of activities in the operating theatre and associated sectors (Article R4311-11 of the French Health Code).

### Preanalytical Work From Sample Collection to Fixation

A first, pre-analytical step is now described, from sample collection in the operating field to placing in a container, in a fresh state or in a preservative liquid (fixing agent). When the practician performs collection by exeresis, the collected sample is often placed in a bowl, such as a kidney dish, given by the surgeon to a circulating IBODE. During this step, an anatomopathological examination form is completed by the nurse, the elements being dictated by the surgeon.

Each collected sample forms biological material that can be sent as such to the anatomopathology laboratory, or distributed into samples before being sent to the anatomopathology laboratory. For this sending, each collection (or sample) is placed in a transport container (e.g. bag, vial, bucket). The transport container is sterile prior to use.

In some organisations, collected samples from different operating rooms are gathered in a common, most often non-sterile, zone in the operating theatre or near the operating theatre.

Surgical specimens with a tumour are preferably sent to the anatomopathology laboratory in fresh condition.

The direct transfer of surgical specimens, from the operating theatre, at room temperature is sometimes implemented. Such a direct transfer is implemented when the anatomopathology laboratory is located close to the operating theatre, the correct storage life of a surgical specimen, at room temperature, being limited to 40 minutes.

In some organisations, samples collected in the operating theatre are placed under vacuum in a pocket of polymeric material, and cooled to about 4° C., for transfer to laboratory. This technique allows the integrity of the sample to be maintained for a limited period of about 72 hours.

Surgical specimens are most often sent to the laboratory ACP after immersion in a preservative liquid (fixing agent). The collected sample or anatomy specimen should be immersed in the fixing agent within a period of time as quick as possible. A ratio of 20 to 50 times the volume of the specimen must be respected. The fixation time depends on the size of the collected sample, and is most often between two hours for a biopsy and 12 to 24 hours for a surgical specimen. Depending on the nature of the collected sample, fixation is achieved by immediate immersion in the fixing agent (for small specimens from biopsy or curettage), or after opening the anatomical specimen (gastrointestinal tract, gallbladder, liver). In some cases, the fixing agent is pre-injected into the anatomical specimen, before being immersed in the fixing agent solution, for example for the bronchi or lungs.

Fixation must occur within a short period of time: otherwise, tissue desiccation or autolysis may make the study difficult or even impossible. Prior to fixation, the hollow organs should be opened and if necessary cleared of their contents, in order to prevent mucosal autolysis. Solid organs such as liver and spleen should be cut into slices to facilitate smooth and rapid penetration of the fixing agent. Many anatomopathology laboratories recommend that tissue collected samples should not be sent fresh, to ensure that the time between surgical resection and fixation by immersion in fixing agent (most often formol) does not exceed one hour for surgical specimens, and ten minutes for biopsies.

Chemical fixation should block endogenous enzymes responsible for the destruction of organites. Chemical fixation should additionally keep cellular, tissue and molecular structures as close to their physiological state as possible. Chemical fixation should also allow immunohistochemical studies and prepare embedding of the sample in an embedding medium, in particular paraffin.

The fixation develops from the surfaces in contact with the fixing agent, so that the fixing agent must be placed first in the container, to avoid the collected sample, for example the anatomical specimen, sticking to the wall of the container. The size of the container must be adapted to the specimen to be fixed. The container may be a vial, a box, or a bucket.

Formol is widely used for preserving collected samples, particularly for morphological analysis and immunohistochemistry. Formol here means an aqueous solution of formic aldehyde, or formaldehyde, (CAS 50-00-0). Formaldehyde is also called methanal, oxomethane, and formol is sometimes called formalin.

Other fixing agents than formol have been provided, for example glyoxal or ethanedial. For example, reference can be made to document EP3416482 (Addax Biosciences, 2018).

However, formaldehyde (in particular buffered formol diluted to 4%) remains the reference in anatomopathology, despite its drawbacks. Standardised immunohistochemistry and in-situ hybridisation tests are validated for collected samples fixed in buffered formol and paraffin-embedded, with patient participation in most international trials conditioned by formol fixation of the tumour sample.

Formaldehyde is marketed as an aqueous solution at concentrations most often between 30% and 55% by weight, the aqueous solutions may contain a formic aldehyde polymerisation inhibitor, such as methanol, at a mass concentration from 0.5 to 15%. Chemical fixing agents based on formaldehyde, in aqueous solution are marketed in three main forms: neutral formol to be diluted in demineralised water, the pH being adjusted with for example sodium hydroxide; buffered formol to be diluted in a buffer solution; neutral buffered formol to be diluted in a buffer solution and to be neutralised with for example calcium carbonate.

Vials, jars and buckets are marketed pre-filled with formol. The vials are for example of polypropylene, with a polyethylene cap, and a capacity of up to, for example, 150 mL. The jars are made of high density polyethylene, with a capacity of half a litre to one litre. The buckets are especially made of polypropylene with a capacity of several litres. When chemically fixing to formol, it is important to ensure constant and complete immersion of the surgical specimens.

During this first, pre-analytical, step of first handling of samples and placing in containers for sending to the ACP laboratory, many errors can be made.

When several sample collections are made for the same patient, identification errors may be made, particularly when collected samples are made from two twin organs such as lungs, kidneys, ovaries, breast or testes. Identification mix-up between the left and right organs can be highly harmful, when only one of the two organs is a carrier of a tumour, or when only one of the two organs is a carrier of a malignant tumour.

When the doctor dictates to the IBODE the elements to complete the pathological examination form, wearing of the surgical mask and the sound environment in the operating theatre can make it difficult to understand. For example, reference may be made to document Magee et al. (Effects of face masks on acoustic analysis and speech perception: Implications for peri-pandemic protocols, J Acoust Soc Am, 2020). This results in the risk of errors in the request that will be sent to the ACP laboratory.

The common ACP examination request form is a form that must be completed by hand. A very great deal of information has to be entered manually. An examination request form includes the patient's identity data, the name of the prescribing physician, the name of the sampler person (if different from that of the prescribing physician), the date and time of the sample collection, collected sample state (fresh or fixed), fixation time, fixing agent used, type of collected sample (biopsy, surgical specimen), the organ to be collected, the location and topography, information for diagnosis (including the patient's history, ongoing medical treatments), the desired number of slides, the existence of an infectious risk.

Filling out forms is often experienced as tedious and repetitive, leading to fatigue and reduced attention, with the risk of forgetting and confusing, fragmented or incorrect information. In particular, the lack of clinical information can be detrimental in anatomopathological interpretation. Furthermore, handwriting is more or less legible, with risks of reading errors in the ACP laboratory.

In some organisations, samples from multiple patients may be on the same cart or bench, to be identified one after the other, leading to risks of identity surveillance errors.

Sometimes complex surgical specimens need to be oriented. For example, a specimen of mastectomy is oriented by the surgeon using wires or clips, when collected. For example, during a breast cancer surgical treatment, for a partial exeresis specimen or a tumorectomy for which a lesion extension assessment is requested, the specimen is oriented in the three planes of space, allowing differential inking of the edges. Information relating to the sampling orientation is not systematically transmitted to the ACP laboratory, or is incomplete.

In order to overcome these drawbacks, according to the invention, an analysis of the video stream is carried out with object identification and tracking, for work carried out from sample collection to placing in a container before sending to the ACP laboratory.

A workstation is placed in proximity to the operating table, this workstation comprising a tray-forming board and means for identifying and tracking objects by analysing a video stream, the samples and the containers for these collected samples being handled on the tray of the workstation. At least one camera films the handling operations performed. Computer vision algorithms analyse the video stream and ensure object identification and tracking.

“Placed in proximity to” here means the fact that the workstation is placed in the operating room or the interventional radiology room or the SIBO hybrid room, the person performing the sample collection preferably placing the collected sample directly on the tray of the workstation.

“Placed in proximity to” here also refers to the case where the workstation is located in a separate room but adjacent (advantageously contiguous) to the operating room, or the interventional radiology room or the SIBO hybrid room. “Placed in proximity to” here means a fixed location for the workstation or a mobile location. Advantageously, the workstation is mounted to a mobile cart, for example a four-wheeled cart.

The analysis of the video stream with identification and tracking of objects advantageously relates to at least one of the following steps: labelling the collected samples, immersing the collected samples in the fixing agent.

Advantageously, for labelling each sample, after verifying the complete identity of the patient, a patient label is edited, this label including the patient's identity (surname, first name, date of birth, gender), as well as the collection date and time.

In some implementations, this label includes the patient number, in alphanumeric and/or coded form (e.g. QR code or Data Matrix). The label is preferably edited by the person who performed the sample collection. The label is placed on the tube, jar or vial to contain the sample.

Analysis of the video stream with identification and tracking of objects, during labelling, makes it possible to warn the operator of a discrepancy between the patient's identity and the data on the ACP order form. Alphanumeric character recognition or reading a unidirectional or bidirectional barcode (QR code, Data Matrix, Aztec, PDF417) gives access to the patient's identity, when analysing the video stream. The ACP analysis request form is placed on the workstation tray, and character recognition is performed.

When data on the ACP request form are not in concordance with the patient's identity, an alarm is generated. According to various embodiments, the alarm is a sound and/or a visual alarm and/or a vibratory alarm.

In some implementations, the alarm includes opening a pop-up window.

Advantageously, the computer vision and video stream analysis algorithms are interfaced with the computerised patient record.

“Computerised patient record” or CPR refers here to the entirety of the health data of a patient managed within a healthcare institution, as well as the software used to organise and upload these data.

The computerised patient record CPR contains, for example, hospital discharge liaison letters, surgical reports, discharge prescriptions, biological reports and imaging reports. The computerised patient record contains different documents required to feed the Shared Medical Record (SMR), or the personal digital space “My health zone”, launched in February 2022, to replace and improve the SMR.

When computer vision and video stream analysis algorithms are interfaced with the computerised patient record CPR, the collection of clinical information is automated, and the prescription of the anatomopathological examination form is simplified. Handwriting is no longer necessary, reducing the risk of errors.

Advantageously, human-machine interface means allow fast navigation in the functionalities of the software, for example touch screen, light pencils, automatic speech recognition, voice synthesis, three-dimensional information visualisation, virtual reality, especially augmented reality, headset or glasses.

In some embodiments, the workstation is provided with stereoscopic, infrared, or laser depth cameras, allowing three-dimensional modelling of the surface of surgical specimens, wherein the obtained model can be saved, oriented, and advantageously annotated digitally.

Advantageously, the workstation is provided with means for digitally annotating the 2D images of the collected samples, and saving them.

The workstation is provided with bar code printing means (one-dimensional or two-dimensional, for example QR code or Data Matrix), the bar code being attached to a container in which the collected sample is placed, a video stream analysis being carried out when printing the bar code, as well as when placing the label on the container.

Advantageously, the ACP examination request form is digital and the identification label placed on the container encrypts the patient's identity and guarantees their anonymity by pseudonymisation. Pseudonymisation refers to measures that ensure protection of personal data, whereby access to this personal data requires resorting to additional information.

Advantageously, the analysis of the video stream makes it possible to control the volume of fixing agent such as formol, and to verify that the volume of fixing agent is adapted to the volume of the surgical specimen to be fixed.

In some implementations, the workstation includes weight sensors, and an amount of fixing agent to be used is communicated to the operator, by a text message or a voice message.

Placing the sample in the fixing agent is filmed and the video stream analysis allows that the appropriate amount of fixing agent is being applied is verified.

The video stream analysis also makes it possible to date start of fixation. An alarm, e.g. an audible alarm and/or displaying a message happens when the fixing agent has been omitted or when the amount of fixing agent is not appropriate.

Advantageously, the operator using the workstation is identified by authentication means, for example with a login and a password and/or facial recognition means, and/or reading a badge, for example RFID or NFC.

### Optional Pre-Analytical Work in a Buffer Zone

A second, optional, pre-analytical step of work carried out in a buffer zone is now described. “Buffer zone” here means a room that can be sterile and is most often non-sterile, preferably placed in proximity to the operating theatre, and in which the collected samples from the operating rooms are gathered, before being transferred to the ACP laboratory.

In this buffer zone, checks are carried out on the ACP request forms, as well as checks on the number of containers (vials, buckets, sachets). Traceability is achieved by handwritten transcription on a paper notebook.

Checks carried out in the buffer zone are repetitive and time-consuming. This results in the risk of human errors, due to a momentary loss of attention or concentration of an operator. These errors may especially relate to the identity of the patient, the nature of the collected sample, the number of containers. When a discrepancy is detected by the operator during checks in the buffer zone, the loss of time caused, for example, by searching for a missing container is a source of annoyance, increasing fatigue and the risk of subsequent errors.

Advantageously, in a method according to the invention, an analysis of the video stream and computer vision algorithms, for example by image segmentation, are implemented during work carried out in the buffer zone.

According to different embodiments, the analysis of the video stream ensures automatic counting of the containers and/or check of the suitability of the fixing agent volume for each collected sample placed in a container (for example by the volume of the collected sample/fixing agent volume ratio, the collected sample being placed in a transparent container).

Advantageously, the absence of fixing agent in a container or the presence of an insufficient amount of fixing agent causes creation of an alarm, for example a text message on a screen and/or an audible alarm.

In some implementations, means make it possible to digitise ACP request forms, for example by automatic character analysis OCR.

Advantageously, the data of the label (e.g. barcode, QR code or Data Matrix) placed on each container are read, the identity of each patient being encrypted, making it possible to preserve anonymity by pseudonymisation.

Advantageously, the identity of the operator working in the buffer zone is checked by authentication means, for example with a login and a password and/or facial recognition means, and/or reading a badge, for example RFID or NFC.

### Work Carried Out at the ACP Laboratory Recording Station

Work carried out at the recording station, at the entrance to the ACP laboratory is now described.

“Recording” here means the workstation receiving the collected samples, delivered for example by a person (such as a courier or the patients themselves), or arriving by automatic transport means (for example by pneumatic transport).

Samples arrive at the recording station from various sources (operating theatres, hospital departments, private clinics), in variable and unpredictable amounts. The recording station receives the entirety of the collected samples from the different prescribers calling on the ACP laboratory.

The recording station is a critical station in the ACP laboratory. In the event of high activity, this station can become a bottleneck, i.e. a station receiving more work requests than its maximum processing capacity can support.

Recording is especially concerned with the identity of the patient and the inventory of the collected samples received. Recording is performed by administrative personnel or a laboratory technician.

When recording, the collected sample is identified by the ACP examination request form. A great deal of information must be checked when recording: patient identity, administrative information, number of containers, description and appearance of the collected samples, clinical information.

When recording, each sample received is assigned an examination number in the computerised laboratory management system LMS and a label with this number is often placed on the container.

The verification work to be carried out at the recording station is repetitive, time-consuming and prone to human error, especially in the event of high traffic, being sources of stress. Traceability is essentially based on the concentration, rigour and vigilance of the personnel working at the recording station.

When an error is detected at this recording station, for example a labelling or recording error in the laboratory management system LMS, corrective actions are time-consuming and tedious, adding fatigue, increasing the risk of subsequent errors.

When examination application forms are incomplete, or difficult to read, personnel of the recording station must attempt to reach the form issuer, especially the prescriber, which takes time and leads to additional risk of errors.

Advantageously, according to the invention, an analysis of the video stream and computer vision algorithms, for example by image segmentation, are implemented during work carried out at the recording station, allowing identification and tracking of objects.

In some implementations, a workstation is placed at the recording station, this workstation comprising a tray on which the containers and collected samples are handled, the tray being equipped with one or more cameras.

In some implementations, one or more cameras placed on the ceiling of the recording station work in concert with cameras of the workstation.

According to different embodiments, the analysis of the video stream ensures automatic counting of the containers and/or check of the suitability of the fixing agent volume for each collected sample placed in a container (for example by the volume of the collected sample/fixing agent volume ratio, the collected sample being placed in a transparent container).

Advantageously, the absence of fixing agent in a container or the presence of an insufficient amount of fixing agent causes creation of an alarm, for example a text message on a screen and/or an audible or vibratory alarm.

In some implementations, means make it possible to digitise ACP request forms, for example by automatic character analysis OCR.

Advantageously, the data of the label (e.g. barcode, QR code or Data Matrix) placed on each container are read, the identity of each patient being encrypted, making it possible to preserve anonymity, by pseudonymisation. The label data advantageously contain the administrative information, description of the collected samples, number of containers and clinical information. Advantageously, reading the bar code is filmed and is the subject of a video stream analysis.

Advantageously, the workstation of the recording station comprises means for printing a barcode (one-dimensional or two-dimensional, for example QR code, Data Matrix), to print a barcode on each container, coding an identification number generated by the laboratory management system LMS. Advantageously, the operations of printing the barcode and fastening this barcode on the container are filmed and are the subject of a video stream analysis.

Advantageously, the identity of the operator working at the recording station is checked by authentication means, for example with a login and a password and/or facial recognition means, and/or reading a badge, for example RFID or NFC.

### Work at the Macroscopy Station

The work carried out at the macroscopy station is now described. In addition to the work described previously with reference to FIGS. 1 to 4, the following provisions are implemented, in various embodiments.

Advantageously, cameras placed above the macroscopy station, for example cameras placed on the ceiling of the room film the work carried out, and an analysis of the video stream from these cameras is carried out.

According to different embodiments, the analysis of the video stream ensures automatic counting of the containers and/or check of the suitability of the fixing agent volume for each collected sample placed in a container (for example by the volume of the collected sample/fixing agent volume ratio, the collected sample being placed in a transparent container).

Advantageously, the absence of fixing agent in a container or the presence of an insufficient amount of fixing agent causes creation of an alarm, for example a text message on a screen and/or an audible alarm.

Advantageously, the identity of the operator working at the macroscopy station is controlled by authentication means, for example with a login and a password and/or facial recognition means, and/or reading a badge, for example RFID or NFC.

### Work at the Dehydration Station

The work carried out at the dehydration station is now described.

At the dehydration station, cassettes from the macroscopic examination are gathered in racks, which can hold up to 150 cassettes, for example.

For example, cassettes are gathered in racks of an automated instrument marketed under the brand name Tissue-Tek VIP, by the company Sakura.

The workstation is a shadow zone in the PCR laboratory workflow: it is indeed impossible to quickly find a sample in a set of cassettes placed at the dehydration station, as all the numbers of the cassettes constituting a batch placed at the dehydration station are unknown.

Advantageously, in a facility according to the invention, one or more cameras film all the cassettes, and the analysis of the video stream makes it possible to read all the numbers of the cassettes, whether this number is alphanumeric or coded, for example, in an OCR code or a Data Matrix.

Advantageously, each cassette bears a number, for example printed on an adhesive medium attached to the cassette, or printed on the cassette. On each cassette, a cassette identifier, for example 24H00000, is also printed, and right next to it, a barcode (for example, Data Matrix) that codes this identifier.

In some implementations, an algorithm first attempts to decode the barcode, and if decoding fails (for example, if the camera focus is poor), a character recognition algorithm (OCR) attempts to decrypt the cassette identifier placed next to the barcode.

In some implementations, one or more cameras are placed on a mobile, motor-driven support, making it possible to carry out travelling.

Advantageously, lighting the cassettes, in particular by LEDs, is carried out.

In some implementations, at least one screen of the dehydration automaton is filmed by one or more cameras, and an analysis of the video stream is performed, for example for character recognition OCR. The information obtained is saved in a database, this information advantageously comprising, for example, the following elements: duration of the dehydration cycle, time remaining until the end of the dehydration cycle, type of cycle.

Advantageously, alarms appearing on the automaton screen are notified in real time to the operator, for example a notification sent by email, by sms, by a pop-up window of a dashboard application.

It is thus possible for an operator away from the automaton to be warned of an operating anomaly of the dehydration automaton, or of an anomaly during the dehydration step.

The video stream analysis can be carried out on each dehydration automaton in the PCR laboratory, these automatons being possibly marketed by different suppliers.

The invention thus provides an agnostic device, indifferent to the models of automatons marketed, the operator using a single human-machine interface for a set of dehydration automatons of different types.

Thus, the operator no longer needs to know in detail the operation and characteristics of each dehydration automaton, one and a single human-machine interface allowing the operator to know the operating state of each automaton in real time.

The operator can be present in the ACP laboratory, or be remote from the ACP laboratory. The operator can receive notifications from automatons placed in different dehydration stations, placed in distinct locations.

### Work at the Embedding or Cryostat Cutting Station

The work carried out at the embedding station is now described, it being understood that what will be described below for the embedding station also applies to the cryostat cutting stations.

The embedding station determines the appearance of the block of embedding material, most often paraffin.

The embedding station is a sensitive station, for the ACP laboratory, for several reasons.

First of all, the work carried out at the embedding station requires manual skill and meticulousness. Certain collected samples are indeed difficult to orient and their orientation may condition the diagnosis, for example skin exeresis, Mohs, pseudo Mohs.

“Mohs” here means a specimen derived from excision by Mohs micrography surgery (MMS). Reference may be made for example to document Harmon et al Mohs micrography surgery, 2022, ISBN 9783132420175. The Mohs technique of excision and pathological examination allows comprehensive marginal analysis combined with reduced sacrifice of healthy tissue. “Pseudo Mohs” or slow Mohs refers here to a sample collection similar to the MMS, followed by paraffin embedding, for a delayed ACP analysis. Reference can be made, for example, to the document Khaddaj, (thèse de médecine, Université de Lille, 2021).

Secondly, there are risks of sample collection loss when pouring from the cassette to the embedding mould.

Advantageously, the embedding automaton is provided with a workstation comprising a barcode scanner, one or more cameras, and a display screen.

In some implementations, one or more endoscopic cameras are placed in proximity to the work top.

In some implementations, one or more cameras are placed on top of the embedding station, for example on the ceiling or on a wall of the PCR laboratory.

When a technician manually enters a cassette, reading its number (e.g. alphanumeric or barcode) is performed. For example, this reading is achieved by means of a scanner or a camera.

Advantageously, when the cassette number is recognised, a software interface opens the patient record and displays macroscopy images. These data make it easier to determine the direction of embedding.

Advantageously, an analysis of the video stream and computer vision algorithms, especially image detection and segmentation, allow counting the collected samples poured into the embedding mould, and check the absence of overlapping of collected samples (for example biopsy core) inside the moulds, or a comparison of the size and surface area, in order to verify that no loss of material has occurred.

In the event of discrepancies in the number of biopsies, an alarm is advantageously generated, this alarm being for example a text or an audible alarm.

### Work at the Cutting and Spreading Station

The work carried out at the cutting and spreading station is now described.

The cutting station and spreading station are sometimes one and a single station, for work carried out by a single technician.

In other implementations, the cutting station is distinct from the spreading station, and work on these two stations is carried out by a same technician or by two different technicians.

At the cutting station, the block of embedding material, usually paraffin, is cut with a microtome into a few micron-thick ribbons. This ribbon is then spread on a glass slide, with or without the help of a water bath, and the slides are then placed on a heating plate.

The cutting and spreading station is a sensitive station in a ACP laboratory. This workstation requires dexterity and consistency in movements.

As the work is repetitive, there is a risk of human error.

In particular, a ribbon from a block may be spread on the wrong slide.

In addition, cutting starts with a step of roughing the block, during which the block may be over-worked, part of the collected sample being then unnecessarily lost.

Conversely, the block may not be worked sufficiently, as the spread ribbons do not allow diagnosis during microscopic observation.

Advantageously, the microtome is provided with a workstation comprising a barcode scanner (one-dimensional or two-dimensional, for example QR code or Data Matrix), one or more cameras and a display screen.

In some implementations, a camera or cameras are aimed at the block inserted into the microtome jaws.

In some implementations, a camera or cameras are aimed at the ribbons.

The screen advantageously displays instructions to be followed for the operator, for example in the form of a text message.

Advantageously, an analysis of the video stream and computer vision algorithms, especially image detection and segmentation, allow tracking of the ribbons to the spreading slides.

Advantageously, detection of the correct position of the ribbon on the slide is carried out, in particular the absence of folds or ridges.

### Work at the Dispensing Station

The work carried out at the dispensing station is now described.

At the dispensing station, the slide trays are prepared in a specific order, with the trays accompanied with examination form in the same order.

Advantageously, in a facility according to the invention, an analysis of the video stream and computer vision algorithms, especially image detection and segmentation, allow counting of the collected samples present on the stained slides, verification of the correlation of this counting with that of the collected samples present in the cassette at the macroscopy station.

In the event of a discrepancy in the counts and/or in the size of the collected samples, an alarm signal is generated, for example a text message and/or an acoustic signal.

For example, when the biopsy core is only 6 mm when it measured 15 mm in macroscopy, a message advises to send the collected sample back to the cuttting-spreading step, stating “please work more block”.

Advantageously, analysis of the video stream and computer vision algorithms, especially image detection and segmentation, allow
verification of the alignment of lamellae and films from the microtome with the glass slides and/or
verification of the staining quality, against staining standards and/or
verification of the correct positioning of the identification labels on the slides.

### Work at the Archiving Station

The work carried out at the archiving station is now described.

At the archiving station, paraffin blocks and glass slides are stored and archived in archive cabinets, in the order of their number assigned at the laboratory, for example 24H00000, and right next to them, a barcode (for example, Data Matrix) that codes this identifier.

Slides and blocks are sorted in chronological order in drawers, according to a storage system per row or per column in the cabinets, for periods ranging from 10 to 30 years in France.

Unarchiving blocks and slides is manual and often untraced, leading to situations where unarchived blocks and slides are missing or lost.

Advantageously, in a facility according to the invention, a station comprising a central processing unit (PC), a touch screen, for example suspended in the immediate vicinity of the archiving cabinets, and a bar code reader makes it possible to identify:
the operator, via for example an identifier and password, or a near-field reading device (NFC), or facial recognition means, or a fingerprint reader;
the block or slide to be archived/unarchived;
the location (for example the coordinates) of the location of the object to be archived (such as a drawer, a cabinet).

An analysis of the video stream and computer vision algorithms, especially image detection and segmentation, allows counting of the unarchived objects, a verification of the correlation of this counting with that of the objects identified on the software.

In the event of a discrepancy in the counts of the unarchived objects compared to the scanned objects, an alarm signal is generated, for example a text message and/or an acoustic signal.

### Advantages of the Invention

The facilities and methods according to the invention have many advantages.

Sample collections performed in the operating theatre follow a complex circuit, including a high number of steps, with a multitude of places (operating theatre, buffer zone, recording station and successive workstations in the ACP laboratory) and actors (prescriber, sampler, transporter, laboratory technicians).

Any failure in this circuit may lead to sample loss, identity errors, or sample orientation errors, inappropriate transport conditions, and sample degradation. These failures may delay or falsely diagnose, or require new sample collection.

The facilities and processes according to the invention improve traceability of the collected samples and reduce the risks of error, at each of the steps and workstations, from the operating field (for example in the operating room) to the analysis of the thin slides under microscopy in the ACP laboratory.

When the surgical specimen is removed by a surgeon and placed in a vial or jar, a camera advantageously films placing the collected sample in a jar or vial, and an algorithm detects the surgical specimen and follows its movement up to the jar or vial.

Advantageously, the algorithm allows checking whether the amount of formol contained in the jar is sufficient to ensure proper tissue conservation.

Advantageously, the surgical specimen is weighed and this weighing is compared to that performed during the surgical sample collection.

The macroscopy station implements at least one identification and tracking algorithm and advantageously at least one character recognition algorithm, making it possible to ensure that the vial or jar placed on the board (associated with a patient, by a unique identifier) corresponds to an operative sample deposited in one or more cassettes.

Thus, the method makes it possible to detect possible human errors in the analysis of an operating collected sample for a patient P. In particular, the method makes it possible to detect incorrect placement of a sample in a cassette,
either by opening a vial not coming from patient P and attempting to deposit this sample in a cassette corresponding to patient P, this error may be called vial mix-up;
or by depositing the sample contained in a vial corresponding to patient P in a cassette not corresponding to patient P, this error being called cassette mix-up.

The method also makes it possible to detect incorrect placement of a sample in a vial or jar, by depositing a sample from a patient P in a labelled vial or jar (for example by Data Matrix) for a patient other than the patient P, this error may be referred to as a patient error.

Furthermore, the method makes it possible to detect the incorrect assignment of a thin slide to a patient.

As has been described, the placement of the sample in a cassette is subject to video stream analysis, and object identification and tracking, as well as recognition of an identifier (for example a Data Matrix type barcode).

The cassette is then embedded in an embedding medium, especially paraffin.

The block obtained is cut with a microtome, the slices obtained, a few microns thick, are then spread on slides. Slides are fixed and then stained to be sent to pathologists for analysis. Slides are paraffin-removed prior to staining, using a solvent such as toluene or xylene. If necessary, immunolabelling is performed in addition to staining, to reveal certain tissue antigens, using labelled antibodies.

These operations are advantageously filmed and an object recognition and object tracking algorithm, as well as an algorithm for recognising an identifier (for example a barcode), makes it possible to ensure that the slides produced bear the identifier present on the cassette.

It is thus possible to ensure that the same identifier, advantageously a unique identifier assigned to a single patient, is present on the vial or jar in which the operating collected sample is placed, during the surgical exeresis or biopsy.

It is thus also possible to ensure traceability of the sample collection performed, from placing it in a vial or jar bearing the identifier, until placing in one or more cassettes, the cassette(s) bearing the same identifier, and from the cassette(s) to the thin slides, each thin slide bearing the same identifier.

Opening and closing the vials, opening and closing the cassettes are advantageously subject to detection and dating (time stamping), in connection with recognition of the identifier of the biological tissue. The method alerts the technician of a loss of concordance between the biological tissue at the end of a processing step (especially placement in a cassette, placement on a thin slide).

The method may advantageously be implemented for small surgical specimens, such as skin exereses, or biopsies.

Advantageously, when implementing the method, the operator can remain focused on the surgical specimen to be analysed, and does not have to be careful to press with the hand or foot on a control member. Possible visual distractions and repeated work interruptions due to the use of hand or foot control members are sources of errors, which can be avoided by the method.

The devices and methods according to the invention can be implemented for existing facilities and equipment, in an agnostic manner, indifferent to the models of automatons marketed (especially dehydration automatons, microtomes, cryostats), the operator advantageously using a single human-machine interface for a set of automatons of different types.

Thus, the devices and methods according to the invention do not impose replacement of the existing machines in the ACP laboratories, with the resulting costs.

The devices and methods according to the invention may be implemented in fully robotic ACP facilities.

Advantageously, however, the invention can be implemented by maintaining manual operations, thus preserving expertise and experience of medical laboratory technicians and physicians qualified in ACP, and reducing the risks of error at each step and station of biological tissue processing.

1. A method for histopathologically examining biological tissues, especially surgical specimen or biopsy, in particular for anatomo-cytopathology ACP, characterised in that it comprises identifying and tracking object by analysing a video stream during a pre-analytical step and/or an analytical step for the biological tissues.

2. The histopathological examination method according to claim 1, characterised in that the pre-analytical step is selected from the group comprising collecting biological tissues from the operating field, orienting the biological tissues, labelling the biological tissues collected, placing the tissues in a container in a fresh state, immersing the biological tissues collected in a preservative liquid such as a fixing agent, preparing a histopathological examination request form, transporting the collected sample to a recording station, recording the collected sample in a laboratory management system.

3. The histopathological examination method according to claim 2, characterised in that it comprises a step of labelling the biological tissues with generating a first patient identity, in alphanumeric form or coded form, in particular by barcode, identifying and tracking object by analysing a video stream comprising a step of reading this first patient identity, in particular reading a barcode.

4. The histopathological examination method according to claim 3, characterised in that it comprises a step of preparing a histopathological examination request form generating a second patient identity, identifying and tracking object by analysing a video stream comprising a step of reading this examination request form and the second patient identity, an alert message being generated in the event of a discrepancy between the first and the second patient identity.

5. The histopathological examination method according to claim 3, characterised in that it comprises a step of querying the computerised patient record and a step of collecting information elements contained in the computerised patient record, for copying into a histopathological examination request form generated in digital form.

6. The histopathological examination method according to claim 1, characterised in that it comprises identifying and tracking object by analysing a video stream when immersing the biological tissues such as a surgical specimen in a preservative liquid such as a fixing agent, an alert message being generated when the volume of the preservative liquid is not adapted to the volume of the biological tissues.

7. The histopathological examination method according to claim 1, characterised in that it comprises a check step carried out in a buffer zone in which a plurality of collected samples of biological tissues, in particular from different operating rooms of an operating theatre, are gathered, the method comprising identifying and tracking object by analysing a video stream for counting the containers placed in the buffer zone.

8. The histopathological examination method according to claim 1, characterised in that it comprises identifying and tracking object by analysing a video stream when recording of a collected sample in a laboratory management system, for counting the containers and/or checking suitability of the fixing agent volume for the collected sample placed in a container.

9. The histopathological examination method according to claim 1, characterised in that it comprises identifying and tracking object by analysing a video stream during a step of preparing biological tissues selected from the group comprising dehydrating, embedding in a material, in particular paraffin, cutting in particular with a cryostat or microtome to form ribbons, spreading the ribbons on supports such as glass slides, dispensing.

10. The histopathological examination method according to claim 9, characterised in that it comprises reading the entirety of the cassette identifiers, these identifiers being alphanumeric or coded, especially in a barcode.

11. The histopathological examination method according to claim 9, characterised in that at least one interface screen of a dehydration machine is filmed, an analysis of a video stream making it possible to generate information comprising the duration of the dehydration cycle, the time remaining until the end of the dehydration.

12. The histopathological examination method according to claim 11, characterised in that it comprises a step of generating an alert message, when an operation alarm has been detected for the dehydration machine, when analysing the video stream from the camera filming the interface screen of the dehydration machine.

13. The histopathological examination method according to claim 1, characterised in that it comprises identifying and tracking object by analysing a video stream during a step of embedding biological tissues in an embedding material such as paraffin, analysing the video stream being able to ensure counting of the biological tissues poured into the embedding mould.

14. The histopathological examination method according to claim 1, characterised in that the analytical step is selected from the group comprising macroscopic study steps.

15. The histopathological examination method according to claim 1, characterised in that it comprises analysing the video streams allowing counting of the collected samples present on the stained slides, from a cassette, and a step of verifying correlation of this number with the number of collected samples present in the cassette.

16. A method for histopathological examination, in particular anatomopathology examination, comprising a step of introducing a tissue into a first container provided with a closure means, a step of closing the first container after introducing the tissue, a step of placing on the first container an identifier of the tissue introduced into the first container, a step of opening the first container and extracting the tissue from the first container, a step of moving at least one part of the tissue extracted from the first container into a second container, the second container bearing an identifier of the part of the tissue, the method being characterised in that it comprises identifying and tracking objects by analysing a video stream, opening of the first container being detected during this identification and tracking of objects, the method comprising a step of detecting and reading the identifier placed on the first container and the identifier placed on the second container, an error message being generated in the event of a discrepancy between the identifier of the first container and the identifier of the second container.

17. The method according to claim 16, characterised in that it comprises a step of recording the instant at which opening of the first container is detected.

18. The method according to claim 16 characterised in that the second container is a cassette, closing the cassette and closing the first container being detected by identifying and tracking objects when analysing the video stream.

19. The method according to claim 16, characterised in that displaying the objects identified when analysing the video stream is carried out, the shape of the objects identified being represented by a bounding window.

20. A device for implementing a method according to claim 1, the device being characterised in that it comprises means for identifying and tracking objects by analysing a video stream from at least one camera.

21. An anatomopathology device, for implementing a method according to claim 16, the device including a workstation comprising a cutting board for supporting a first container provided with a closure means and a second container, the device comprising means for identifying and tracking objects by analysing a video stream, these means being able to detect opening of the first container, the device comprising means for detecting and reading an identifier placed on the first container and an identifier placed on the second container, the device comprising means for generating an error message in the event of a discrepancy between the identifier of the first container and the identifier of the second container.

22. The device according to claim 21, characterised in that the cutting board comprises a recess capable of housing a first or second container, the first container preferably being a vial, the second container preferably being a cassette.

23. The device according to claim 21, characterised in that the detection and reading means comprise a one-or two-dimensional barcode reader.

24. The device according to claim 21, characterised in that the workstation comprises weighing means.

25. The device according to claim 21, characterised in that the workstation comprises measuring means along several axes.

26. The device according to claim 21, characterised in that it comprises a biological tissue embedding station to form a block, a station for cutting the block into thin slices with a microtome, and a station for spreading the slices on slides, the device comprising means for printing an identifier of the biological tissue on the block and on the slides.
